(19) United States
(12) Patent Application Publication
(10) Pub. No.: US 2026/0188029 A1
(43) Pub. Date: Jul. 2, 2026

(54) CELL COUNTING METHOD AND APPARATUS, DEVICE, STORAGE MEDIUM, AND PROGRAM PRODUCT

(71) Applicants: Wenzhou University of Technology, Wenzhou City (CN); Wenzhou Medical University, Wenzhou City (CN)

(72) Inventors: Yuanyuan WANG, Wenzhou City (CN); Zuoping Tan, Wenzhou City (CN); Caiye Fan, Wenzhou City (CN); Xudong Wang, Wenzhou City (CN); Yikui Zhang, Wenzhou City (CN)

(21) Appl. No.: 19/329,192

(22) Filed: Sep. 15, 2025

(30) Foreign Application Priority Data
Dec. 31, 2024 (CN) 202411987291.8

(51) Int. Cl. G06V 20/69; G06T 7/00; G06V 10/77; G06V 10/80; G06V 10/82; G16H 30/20

(52) U.S. Cl. CPC G06V 20/698; G06T 7/0012; G06V 10/7715; G06V 10/806; G06V 10/82; G06V 20/693; G06V 20/695; G16H 30/20; G06T 2207/30024

(57) ABSTRACT

Provided are a cell counting method and apparatus, a device, a storage medium, and a program product, relating to the technical field of image processing. The method includes: acquiring a cell image and cell labeling information in one-to-one correspondence with the cell image; inputting the cell image into a pre-constructed cell prediction model to obtain predicted cell point information corresponding to the cell image, where the predicted cell point information includes predicted cell point position information and confidence values in one-to-one correspondence with predicted cell points; performing one-to-one matching on reference points corresponding to the cell image with the predicted cell points based on the cell labeling information and the predicted cell point information to obtain a matching result; and adjusting the cell prediction model based on the matching result, where the cell prediction model is used for predicting cell positions and cell counts.

### CROSS REFERENCE TO RELATED APPLICATION

This patent application claims the benefit and priority of Chinese Patent Application No. 202411987291.8, filed with the China National Intellectual Property Administration on Dec. 31, 2024, the disclosure of which is incorporated by reference herein in its entirety as part of the present application.

### TECHNICAL FIELD

The present disclosure relates to the technical field of image processing, and specifically, to a cell counting method and apparatus, a device, a storage medium, and a program product.

### BACKGROUND

Cell counting is a critical issue in medical image research, where accurate cell counting can reliably indicate potential cellular diseases and related pathological changes.

Currently, existing cell counting methods primarily include density map-based cell counting methods and cell counting methods based on pseudo-bounding box localization. The density map-based counting method employs density map regression to achieve cell counting, i.e., it uses pixel-level density map regression to directly predict a density value of each pixel and then sums the density values across the entire image to obtain a total cell count. However, density map-based cell counting methods cannot provide precise locations of individual cells, making subsequent cell analysis tasks unfeasible. The cell counting method based on pseudo-bounding box localization primarily predicts locations of individual cells for counting. This method relies on generated pseudo-bounding boxes to accomplish cell counting and localization. When cells are highly crowded or overlapping, the cell counting method based on pseudo-bounding box localization may misidentify multiple cells as a single cell.

In view of this, there is an urgent need for a method that can both precisely locate individual cells and accurately predict cell counts.

### SUMMARY

Accordingly, the present disclosure provides a cell counting method and apparatus, a device, a storage medium, and a program product, to precisely locate individual cells while accurately predict a cell count.

According to a first aspect, the present disclosure provides a cell counting method, including:
acquiring a cell image and cell labeling information in one-to-one correspondence with the cell image, where the cell labeling information includes pre-labeled reference point position information and a cell count;
inputting the cell image into a pre-constructed cell prediction model to obtain predicted cell point information corresponding to the cell image, where the predicted cell point information includes predicted cell point position information and confidence values in one-to-one correspondence with predicted cell points;
performing one-to-one matching on reference points corresponding to the cell image with the predicted cell points based on the cell labeling information and the predicted cell point information to obtain a matching result; and
adjusting the cell prediction model based on the matching result, where the cell prediction model is used for predicting cell positions and cell counts.

In the present disclosure, a set of annotated point-label maps is received for training, and a set of predicted cell points, including predicted cell point position information and a cell count, are directly generated from the cell image during inference. By training the cell prediction model, both cell counting and localization are achieved simultaneously, eliminating redundant intermediate representation steps and improving localization accuracy and counting performance.

In an optional implementation, the step of performing one-to-one matching on reference points corresponding to the cell image with the predicted cell points based on the cell labeling information and the predicted cell point information includes:
calculating offsets between the predicted cell points and the reference points based on the predicted cell point position information, the reference point position information, and the confidence values in one-to-one correspondence with the predicted cell points, to obtain an offset matrix; and
minimizing the offsets between the reference points and the predicted cell points using a Hungarian algorithm based on the offset matrix, and determining the reference points in one-to-one correspondence with the predicted cell points.

In this implementation, the one-to-one matching approach not only avoids two unintended scenarios: matching multiple reference points to a single predicted point, and matching multiple predicted points to a single reference point, but also improves matching accuracy between predicted cell points and reference points, thereby enhancing the precision of cell prediction results.

In an optional implementation, the offsets between the predicted cell points and the reference points are calculated using the following formula:

$$D ( P , \hat{P} ) = {( τ {‖ {p}_{i} - ‖}_{2} - )}_{i ∈ N , j ∈ M}$$

where P represents the reference points, \hat{P} represents the predicted cell points, pᵢ represents the reference point position information of an i-th reference point, [symbol] represents the predicted cell point position information of a j-th predicted cell point, [symbol] represents the confidence value of the j-th predicted cell point, ∥·∥2 represents a distance between the i-th reference point and the j-th predicted cell point, τ represents a weight, N represents a total number of the reference points, and M represents a total number of the predicted cell points.

In this implementation, by calculating the offsets between the predicted cell points and the reference points, the accuracy of cell detection results can be effectively quantified, thereby optimizing model performance. This method not only facilitates identification of cell position errors but also weights high-quality predictions based on confidence values, thereby enhancing model stability and accuracy in high-density cell scenarios.

In an optional implementation, cell prediction model includes:
a pre-trained convolutional neural network, a feature pyramid network, and a decoding network connected sequentially;
where an output end of the decoding network is further connected to a regression branch network and a classification branch network.

In this implementation, the use of a pre-trained convolutional neural network for feature extraction, combined with the architecture integrating a feature pyramid network and a decoding network, enables full utilization of multi-scale image features. This configuration enhances the accuracy of cell detection, thereby effectively improving the precision of both cell counting and localization.

In an optional implementation, the step of inputting the cell image into the pre-constructed cell prediction model to obtain the predicted cell point information corresponding to the cell image includes:
extracting first image features from the cell image using the pre-trained convolutional neural network;
performing feature upsampling and feature fusion on the first image features using the feature pyramid network to obtain second image features;
performing feature decoding on the second image features using the decoding network to generate a feature map; and
inputting the feature map into the regression branch network and the classification branch network, where the regression branch network is used for outputting the predicted cell point position information, and the classification branch network is used for outputting the confidence values in one-to-one correspondence with each predicted cell point.

In this implementation, the pre-trained convolutional neural network extracts image feature, effectively capturing critical information from the cell image; the feature pyramid network performs feature upsampling and fusion, enabling full utilization of multi-scale features; the decoding network generates a feature map to enhance the accuracy of subsequent regression and classification results; the regression branch network and the classification branch network output predicted cell point position information and confidence values, respectively, thereby ensuring precise prediction of both cell positions and counts.

According to a second aspect, the present disclosure provides a cell counting method, including:
acquiring an image of cells to be counted; and
inputting the image of cells to be counted into the cell prediction model of the cell counting method described above, to output a cell count in the image of cells to be counted and cell positions in one-to-one correspondence with cells.

According to a third aspect, the present disclosure provides a cell counting apparatus, including:
an acquisition module configured to acquire a cell image and cell labeling information in one-to-one correspondence with the cell image, where the cell labeling information includes pre-labeled reference point position information and a cell count;
a prediction module configured to input the cell image into a pre-constructed cell prediction model to obtain predicted cell point information corresponding to the cell image, where the predicted cell point information includes predicted cell point position information and confidence values in one-to-one correspondence with predicted cell points;
a matching module configured to perform one-to-one matching on reference points corresponding to the cell image with the predicted cell points based on the cell labeling information and the predicted cell point information to obtain a matching result; and
an optimization module configured to adjust the cell prediction model based on the matching result, where the cell prediction model is used for predicting cell positions and cell counts.

According to a fourth aspect, the present disclosure provides a computer device, including a memory and a processor, where the memory and the processor are communicatively connected to each other; the memory stores computer instructions, and the processor executes the computer instructions to perform the cell counting method according to the first aspect or any implementation thereof.

According to a fifth aspect, the present disclosure provides a computer-readable storage medium, storing computer instructions, where the computer instructions are used to cause a computer to perform the cell counting method according to the first aspect or any implementation thereof.

According to a sixth aspect, the present disclosure provides a computer program product, including computer instructions, where the computer instructions are used to cause a computer to perform the cell counting method according to the first aspect or any implementation thereof.

It should be noted that, the cell counting apparatus, computer device, computer-readable storage medium, and computer program product provided by the present disclosure correspond to the aforementioned cell counting method. Therefore, for the beneficial effects of the cell counting apparatus, computer device, computer-readable storage medium, and computer program product, reference can be made to the corresponding descriptions of the beneficial effects of the cell counting method above, and details will not be repeated herein.

### BRIEF DESCRIPTION OF THE DRAWINGS

To describe the technical solutions in the specific implementations of the present disclosure or the prior art more clearly, the accompanying drawings required for describing the specific implementations or the prior art are briefly described below. Apparently, the accompanying drawings in the following description show merely some implementations of the present disclosure, and a person of ordinary skill in the art may still derive other accompanying drawings from these accompanying drawings without creative efforts.

FIG. 1 is a schematic flowchart of a cell counting method according to an embodiment of the present disclosure;

FIG. 2 is a schematic diagram illustrating matching of reference points and predicted points according to an embodiment of the present disclosure;

FIG. 3 is a schematic structural diagram of a cell prediction model according to an embodiment of the present disclosure;

FIG. 4 is a schematic diagram illustrating cell counting results according to an embodiment of the present disclosure;

FIG. 5 is a structural block diagram of a cell counting apparatus according to an embodiment of the present disclosure; and

FIG. 6 is a schematic diagram of a hardware structure of a computer device according to an embodiment of the present disclosure.

### DETAILED DESCRIPTION OF THE EMBODIMENTS

In order to make the objectives, technical solutions, and advantages of the embodiments of the present disclosure clearer, the technical solutions in the embodiments of the present disclosure will be clearly and completely described below in conjunction with the accompanying drawings in the embodiments of the present disclosure. Apparently, the described embodiments are some, rather than all of the embodiments of the present disclosure. All other embodiments obtained by those skilled in the art based on the embodiments of the present disclosure without creative efforts shall fall within the protection scope of the present disclosure.

According to the embodiments of the present disclosure, an embodiment of a cell counting method is provided. It should be noted that, steps shown in the flowchart in the accompanying drawings may be executed in a computer system such as a set of computer executable instructions. Moreover, although a logic sequence is shown in the flowchart, the shown or described steps may be executed in a sequence different from that described here.

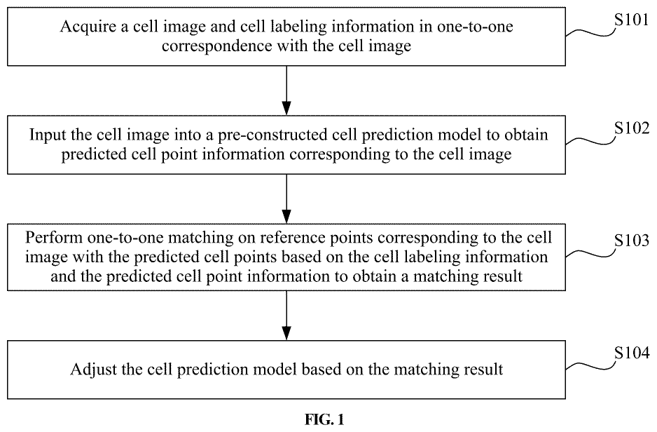

This embodiment provides a cell counting method, which may be executed by devices such as a server, a terminal, or a mobile terminal. FIG. 1 is a flowchart of a cell counting method according to an embodiment of the present disclosure. As shown in FIG. 1, the process includes the following steps:

Step S101: Acquire a cell image and cell labeling information in one-to-one correspondence with the cell image, where the cell labeling information includes pre-labeled reference point position information and a cell count. The cell image may be obtained through devices such as microscopes, and the reference point position information may be pre-marked manually or automatically by users based on the cell image, including cell position information and a cell count.

Step S102: Input the cell image into a pre-constructed cell prediction model to obtain predicted cell point information corresponding to the cell image, where the predicted cell point information includes predicted cell point position information and confidence values in one-to-one correspondence with predicted cell points.

In this embodiment, the cell prediction model may include a pre-trained convolutional neural network, a feature pyramid network, and a decoding network. These networks extract features from the cell image and output the predicted cell point position information and confidence values through a regression branch network and a classification branch network.

Step S103: Perform one-to-one matching on reference points corresponding to the cell image with the predicted cell points based on the cell labeling information and the predicted cell point information to obtain a matching result.

In this embodiment, after preliminary prediction is completed using the pre-constructed cell prediction model, the accuracy of the predicted points is further verified based on the cell labeling information and the predicted cell point information. Specifically, a Hungarian algorithm may be used for optimal one-to-one matching. Unmatched reference points are temporarily retained, and the matched reference points and predicted points are dynamically updated. That is, predicted points with potential and better performance are pushed toward corresponding targets. Eventually, the one-to-one matching process gradually determines the final predicted points, avoiding undercounting and overcounting issues. This improves the normalized average precision metric, enabling more accurate inference of cell positions and cell counts.

Step S104: Adjust the cell prediction model based on the matching result, where the cell prediction model is used for predicting cell positions and cell counts.

The matching result includes a one-to-one matching relationship between reference points and predicted cell points, as well as a matching count. The cell prediction model is then optimized based on the matching result. Specifically, losses between the predicted points and the reference points are calculated based on the matching result, including regression losses and classification losses. The computed losses are combined into a total loss, and a gradient of the loss is calculated using a backpropagation algorithm to update model weights and biases, thereby optimizing the cell prediction model. After the model is updated, the same cell image may be used again for prediction to test the improvement of the model.

In this embodiment, the loss function consists of two parts: classification loss and regression loss. The classification loss is used to train the classification of predicted points, while the regression loss is used to guide the regression of point coordinates. A final loss function [symbol] is a sum of the classification loss [symbol] and the regression loss [symbol] , as shown in the following formulas:

$${ℒ}_{c l s} = - \frac{1}{M} \{ {\sum }_{i = 1}^{N} log {\hat{c}}_{ε ( i )} + {λ}_{1} {\sum }_{i = N + 1}^{M} log ( 1 - {\hat{c}}_{ε ( i )} ) \}$$

$${ℒ}_{loc} = \frac{1}{N} {\sum }_{i = 1}^{N} {‖ {p}_{i} - {\hat{p}}_{ε ( i )} ‖}_{2}^{2}$$

$$ℒ = {ℒ}_{c l s} + {λ}_{2} {ℒ}_{loc}$$

where N represents a total number of reference points; M represents a total number of predicted cell points; both i and j are variables; both λ₁ and λ₂ are weights; Ĉ_{ε(i)} represents a confidence value of a predicted cell point matched with an i-th reference point; \hat{p}_{ε(i)} represents position information of the predicted cell point matched with the i-th reference point; pᵢ represents reference point position information of the i-th reference point.

In this embodiment, a set of annotated point-label maps is received for training, and a set of predicted cell points, including predicted cell point position information and a cell count, are directly generated from the cell image during inference. By training the cell prediction model, both cell counting and localization are achieved simultaneously, eliminating redundant intermediate representation steps and improving localization accuracy and counting performance.

In some optional implementations, the step of performing one-to-one matching on reference points corresponding to the cell image with the predicted cell points based on the cell labeling information and the predicted cell point information includes:
calculating offsets between the predicted cell points and the reference points based on the predicted cell point position information, the reference point position information, and the confidence values in one-to-one correspondence with the predicted cell points, to obtain an offset matrix; and
minimizing the offsets between the reference points and the predicted cell points using a Hungarian algorithm based on the offset matrix, and determining the reference points in one-to-one correspondence with the predicted cell points.

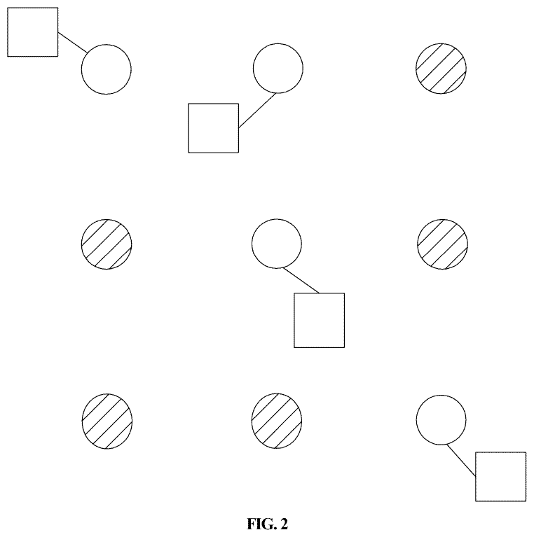

In this embodiment, the Hungarian algorithm may be employed as a matching strategy to perform one-to-one matching between predicted points (hollow circles) and pre-annotated reference points (square boxes), as illustrated in FIG. 2. In other words, the one-to-one matching strategy assigns a reference point to each predicted point.

The offset matrix in this embodiment is an N×M pairwise matching cost matrix that measures the distance between each pair of points. The Hungarian algorithm is applied to process the obtained offset matrix. As an optimization algorithm, the Hungarian algorithm can solve bipartite graph matching problems in polynomial time. The Hungarian algorithm can minimize the total offset between predicted cell points and reference points in the offset matrix, thereby determining the corresponding reference point for each predicted cell point. This ensures an optimal matching result, enhancing the accuracy of matching between the predicted cell points and the reference points.

In this embodiment, the one-to-one matching approach not only avoids two unintended scenarios: matching multiple reference points to a single predicted point, and matching multiple predicted points to a single reference point, but also improves matching accuracy between predicted cell points and reference points, thereby enhancing the precision of cell prediction results.

In some optional implementations, the offsets between the predicted cell points and the reference points are calculated using the following formula:

$$D ( P , \hat{P} ) = {( τ {‖ {p}_{i} - \hat{{p}_{J}} ‖}_{2} - \hat{{c}_{J}} )}_{i ∈ N , j ∈ M}$$

where P represents the reference points, \hat{P} represents the predicted cell points, pᵢ represents the reference point position information of an i-th reference point,

$$\hat{{p}_{{J}_{j}}}$$

represents the predicted cell point position information of a j-th predicted cell point,

$$\hat{{c}_{J}}$$

represents the confidence value of the j-th predicted cell point, ∥·∥2 represents a distance between the i-th reference point and the j-th predicted cell point, τ represents a weight, N represents a total number of the reference points, and M represents a total number of the predicted cell points.

After the feature map is obtained through feature extraction, each pixel on the feature map corresponds to an s×s region in the input image. Within this region, a set of fixed reference points R={Rₖ|k ∈{1, . . . , K}} is predefined first, with positions Rₖ=(xₖ, yₖ). These reference points can be arranged in this region. Since each location on the feature map has K reference points, the regression branch generates a total of H×W×K predicted points. Given an offset

$$( {Δ}_{{jx}^{′}}^{k} , {Δ}_{{jy}^{′}}^{k} )$$

between reference point Rₖ and corresponding predicted point \hat{p}ⱼ=(xⱼ, yⱼ), the coordinates of predicted point \hat{p}ⱼ are computed as follows:

$$\hat{{x}_{J}} = {x}_{k} + {γΔ}_{{jx}^{′}}^{k}$$

$$\hat{{y}_{J}} = {y}_{k} + {γΔ}_{{jy}^{′}}^{k}$$

where γ is a normalization term to scale offsets for correcting relatively minor predictions.

In this embodiment, by calculating the offsets between the predicted cell points and the reference points, the accuracy of cell detection results can be effectively quantified, thereby optimizing model performance. This method not only facilitates identification of cell position errors but also weights high-quality predictions based on confidence values, thereby enhancing model stability and accuracy in high-density cell scenarios.

In some optional implementations, the cell prediction model includes:
a pre-trained convolutional neural network, a feature pyramid network, and a decoding network connected sequentially;
where an output end of the decoding network is further connected to a regression branch network and a classification branch network.

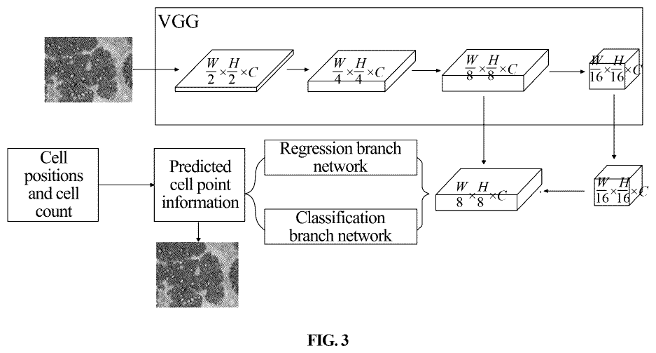

The structure of the cell prediction model is shown in FIG. 3. A pre-trained VGG-16 convolutional neural network can be used to extract deep image features. Then, feature upsampling and lateral connections are implemented through a feature pyramid network (FPN) structure. A feature map generated after feature decoding is then input into both the regression branch network and the classification branch network, ultimately outputting predicted cell point information.

In this embodiment, the use of a pre-trained convolutional neural network for feature extraction, combined with the architecture integrating a feature pyramid network and a decoding network, enables full utilization of multi-scale image features. This configuration enhances the accuracy of cell detection, thereby effectively improving the precision of both cell counting and localization.

In some optional implementations, the step of inputting the cell image into the pre-constructed cell prediction model to obtain the predicted cell point information corresponding to the cell image includes:
extracting first image features from the cell image using the pre-trained convolutional neural network;
performing feature upsampling and feature fusion on the first image features using the feature pyramid network to obtain second image features;
performing feature decoding on the second image features using the decoding network to generate a feature map; and
inputting the feature map into the regression branch network and the classification branch network, where the regression branch network is used for outputting the predicted cell point position information, and the classification branch network is used for outputting the confidence values in one-to-one correspondence with each predicted cell point.

In this embodiment, a pre-trained VGG-16 convolutional neural network can be used to extract deep image features. These features are then upsampled and laterally connected through a feature pyramid network (FPN) structure, with a decoding process of the FPN being implemented by a decoding network. The decoding network includes three main convolutional layers and upsampling operations to fuse detailed information from low-level (high-resolution) feature maps with semantic information from high-level (low-resolution) feature maps, ultimately producing a fine-grained feature map. The cell prediction model also includes two main parallel branches: a regression branch network and a classification branch network.

Specifically, the regression branch is used to predict coordinates of predicted cell points. It processes the feature maps output by the FPN through four convolutional layers, and finally outputs the coordinates of each predicted cell point through one convolutional layer as a tensor with shape (batch_size, num_anchors, 2), representing the coordinates of each predicted cell point. The classification branch predicts the class confidence of cells (where confidence determines which predicted cell points are valid and which can be ignored). Similarly, the classification branch processes the feature maps output by the FPN through four convolutional layers, and finally uses a Softmax function to output a tensor with shape (batch_size, num_anchors, num_classes), representing a class probability for each predicted cell point. The complete model output is information containing both prediction confidence and coordinates, that is, two parallel branches are used to predict a set of predicted cell point position information and corresponding confidence values.

Additionally, in this embodiment, an evaluation metric called nAP (density-normalized) is further defined based on average precision (where the average precision is the area under a precision-recall (PR) curve) to assess localization errors and counting performance. The formula is as follows:

$$1 ( \hat{{p}_{J}} , {p}_{i} ) = \{ \begin{matrix}1 , & if d ( \hat{{p}_{J}} , {p}_{i} ) / dkNN ( {p}_{i} ) < δ , \\ 0 , & otherwise\end{matrix} ,$$

where d( [symbol] , pᵢ) is ∥ [symbol] −pᵢ∥₂, representing a Euclidean distance, dkNN(pᵢ) represents an average distance to the k nearest neighbors of pᵢ, and threshold δ controls localization accuracy. Then, feature extraction is performed: Based on VGG16, downsampled feature extraction is performed using the first 13 convolutional layers of VGG to extract deep image features. This process includes four stages, sequentially producing feature maps of sizes (H/2, W/2), (H/4, W/4), (H/8, W/8), and (H/16, W/16) through convolutional operations. These features are then upsampled using nearest-neighbor interpolation to obtain more refined feature maps.

Furthermore, Fs is used to denote a deep feature map output from a backbone network, where s represents a downsampling stride, and Fs has a size of H×W. Based on Fs, two parallel branches (classification branch and regression branch) are employed for point coordinate regression and predicted point classification. Both branches consist of three stacked convolutional layers with ReLU activation functions introduced between layers. For the classification branch, it outputs confidence values after Softmax normalization to determine which predicted points are valid and which can be ignored. For the regression branch, leveraging the inherent translation invariance property of convolutional layers, it predicts offsets of point coordinates based on given reference points. The regression branch can perform prediction for a large number of predicted points, which are dynamically updated through one-to-one matching, with best-performing predicted points selected as final predicted points.

Additionally, when training the cell prediction model, the present disclosure can use a set of labeled reference points as learning targets to provide exact cell positions and cell counts in cell images. Specifically, given a cell image containing N cells, pᵢ=(xᵢ, yᵢ), i∈{1, . . . , N} is used to represent a position of an i-th cell, i.e., reference point i is located at (xᵢ, yᵢ). A set containing all cell points can be further represented as P={pᵢ|i∈{1, . . . , N}}, where N represents a total number of reference points. Furthermore, this point set is used to predict two other sets: \hat{P}={ [symbol] |j∈{1, . . . , M}} and Ĉ={ [symbol] |j∈{1, . . . , M}}, where M represents a total number of predicted points (i.e., predicted total cell count), [symbol] denotes positions of the predicted points, and [symbol] denotes confidence values of the predicted points. During prediction, it is necessary to ensure that the distance between [symbol] and pi is as close as possible, with sufficiently high confidence values [symbol] . Additionally, the predicted cell count M should closely match the actual count N. After prediction, the final output includes predicted cell point information.

In this embodiment, the pre-trained convolutional neural network extracts image feature, effectively capturing critical information from the cell image; the feature pyramid network performs feature upsampling and fusion, enabling full utilization of multi-scale features; the decoding network generates a feature map to enhance the accuracy of subsequent regression and classification results; the regression branch network and the classification branch network output predicted cell point position information and confidence values, respectively, thereby ensuring precise prediction of both cell positions and counts.

This embodiment further provides a cell counting method, which may be executed by devices such as a server, a terminal, or a mobile terminal. The process includes the following steps:

Step S201: Acquire an image of cells to be counted.

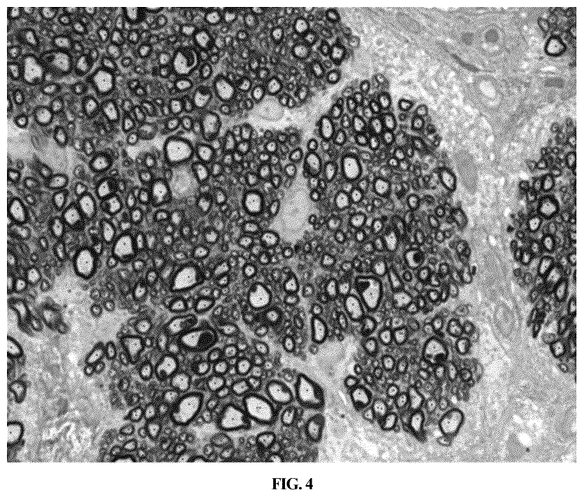

Step S202: Input the image of cells to be counted into the cell prediction model of the cell counting method described in any one of the foregoing embodiments, to output a cell count in the image of cells to be counted and cell positions in one-to-one correspondence with cells. The cell counting effect is illustrated in FIG. 4.

Further functional descriptions of each step are consistent with the corresponding embodiments mentioned above and will not be reiterated here.

In this embodiment, the direct use of point labels as learning targets enables not only accurate inference of cell counts in images but also outputs precise point locations to localize individual cells. This approach facilitates more comprehensive cell analysis.

This embodiment further provides cell counting apparatus, for implementing the foregoing embodiments and preferred implementations, which have been illustrated and are not described again. As used below, the term “module” may implement the combination of software and/or hardware having predetermined functions. Although the apparatus described in the following embodiments is preferably implemented by software, implementation by hardware or the combination of the software and the hardware is also possible and may be conceived.

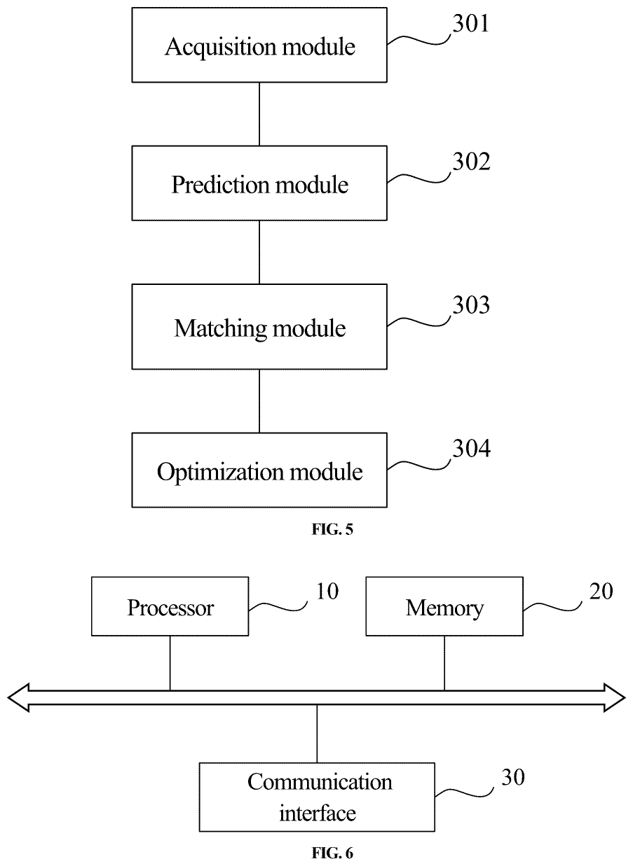

This embodiment provides a cell counting apparatus. As shown in FIG. 5, the apparatus includes:
an acquisition module 301 configured to acquire a cell image and cell labeling information in one-to-one correspondence with the cell image, where the cell labeling information includes pre-labeled reference point position information and a cell count;
a prediction module 302 configured to input the cell image into a pre-constructed cell prediction model to obtain predicted cell point information corresponding to the cell image, where the predicted cell point information includes predicted cell point position information and confidence values in one-to-one correspondence with predicted cell points;
a matching module 303 configured to perform one-to-one matching on reference points corresponding to the cell image with the predicted cell points based on the cell labeling information and the predicted cell point information to obtain a matching result, which specifically includes calculating offsets between the predicted cell points and the reference points based on the predicted cell point position information, the reference point position information, and the confidence values in one-to-one correspondence with the predicted cell points, to obtain an offset matrix; and minimizing the offsets between the reference points and the predicted cell points using a Hungarian algorithm based on the offset matrix, and determining the reference points in one-to-one correspondence with the predicted cell points; and
an optimization module 304 configured to adjust the cell prediction model based on the matching result, where the cell prediction model is used for predicting cell positions and cell counts.

In some optional implementations, the prediction module 302 includes:
a feature extraction module configured to extract first image features from the cell image using a pre-trained convolutional neural network; perform feature upsampling and feature fusion on the first image features using a feature pyramid network to obtain second image features; perform feature decoding on the second image features using a decoding network to generate a feature map; and input the feature map into the regression branch network and the classification branch network, where the regression branch network is used for outputting the predicted cell point position information, and the classification branch network is used for outputting the confidence values in one-to-one correspondence with each predicted cell point.

The cell counting apparatus in this embodiment is presented in the form of functional units. Here, a “unit” refers to an application-specific integrated circuit (ASIC), a processor and memory executing one or more software programs or fixed programs, and/or other components capable of providing the aforementioned functionalities.

Further functional descriptions of each module and unit are consistent with the corresponding embodiments mentioned above and will not be reiterated here.

An embodiment of the present disclosure further provides a computer device, which has the cell counting apparatus shown in FIG. 5.

Refer to FIG. 6, which is a schematic structural diagram of a computer device according to an optional embodiment of the present disclosure. As shown in FIG. 6, the computer device includes: one or more processors 10, a memory 20, and interfaces for connecting various components, including high-speed and low-speed interfaces. The components are communicatively connected to each other by using different buses, and can be installed on a common mainboard or installed in other ways as required. The processor can process instructions executed within the computer device, including instructions stored in or on the memory to display graphical information of a graphical user interface (GUI) on an external input/output apparatus (such as a display device coupled to an interface). In some optional implementations, multiple processors and/or buses may be used with multiple memories if required. Similarly, multiple computer devices may be interconnected, with each device providing part of the necessary operations (e.g., as a server array, a blade server group, or a multi-processor system). FIG. 6 illustrates an example with one processor 10.

The processor 10 may be a central processing unit (CPU), a network processor, or a combination thereof. The processor 10 may further include a hardware chip. The hardware chip may be an ASIC, a programmable logic device, or a combination thereof. The programmable logic device may be a complex programmable logic device, a field-programmable gate array, a generic array logic, or any combination thereof.

The memory 20 stores instructions executable by at least one processor 10 to enable the at least one processor 10 to perform the methods illustrated in the aforementioned embodiments.

The memory 20 may mainly include a program storage area and a data storage area. The program storage area may store an operating system, and applications required for at least one function; and the data storage area may store data that is created based on the use of the computer device. Moreover, the memory 20 may further include a high-speed random access memory and a non-transitory memory, such as at least one disk storage device, a flash memory device, or other non-transitory solid-state memory devices. In some optional implementations, the memory 20 may include a memory remotely disposed for the processor 10. The remote memory may be connected to the computer device via a network. Examples of the foregoing network include, but are not limited to, the Internet, an enterprise intranet, a local area network, a mobile communication network, and a combination thereof.

The memory 20 may include a volatile memory, such as a random access memory (RAM) and a non-volatile memory such as a flash memory, a hard disk drive (HDD), or a solid-state drive (SSD). The memory 20 may also include a combination of the above memory types.

The computer device further includes a communication interface 30 for enabling the computer device to communicate with other devices or communication networks.

An embodiment of the present disclosure also provides a computer-readable storage medium. The methods according to the embodiments of the present disclosure may be implemented in hardware, firmware, or as computer code recorded on a storage medium or downloaded over a network, where the code is originally stored in a remote storage medium or non-transitory machine-readable storage medium and subsequently stored in a local storage medium. Thus, the methods described herein may be stored as software on a storage medium for use with general-purpose computers, dedicated processors, or programmable or specialized hardware. The storage medium may be a magnetic disk, an optical disc, a read-only memory (ROM), a RAM, a flash memory, an HDD, an SSD, or the like. The storage medium may alternatively be a combination of the above memory types. It should be understood that computers, processors, microprocessor controllers, or programmable hardware include storage components capable of storing or receiving software or computer code. When the software or computer code is accessed and executed by the computer, processor, or hardware, the methods illustrated in the above embodiments are implemented.

A portion of the present disclosure can be applied as a computer program product, such as a computer program instruction, which, when executed by a computer, can invoke or provide the methods and/or technical solutions according to the present disclosure through the operation of the computer. A person skilled in the art should understand that the presence of computer program instructions in a computer-readable medium includes, but is not limited to, source files, executable files, installation package files, etc. Accordingly, the execution of computer program instructions by a computer includes, but is not limited to: direct execution of the instructions by the computer, compilation of the instructions and execution of the compiled program by the computer, reading and execution of the instructions by the computer, or reading and installation of the instructions and execution of the installed program by the computer. The computer-readable medium may be any available computer-readable storage medium or communication medium accessible to the computer.

Although the embodiments of the present disclosure are described with reference to the accompanying drawings, those skilled in the art may make various modifications and variations without departing from the spirit and scope of the present disclosure. These modifications and variations shall fall within the scope defined by the claims.

What is claimed is:

1. A cell counting method, comprising:
acquiring a cell image and cell labeling information in one-to-one correspondence with the cell image, wherein the cell labeling information comprises pre-labeled reference point position information and a cell count;
inputting the cell image into a pre-constructed cell prediction model to obtain predicted cell point information corresponding to the cell image, wherein the predicted cell point information comprises predicted cell point position information and confidence values in one-to-one correspondence with predicted cell points;
performing one-to-one matching on reference points corresponding to the cell image with the predicted cell points based on the cell labeling information and the predicted cell point information to obtain a matching result; and
adjusting the cell prediction model based on the matching result, wherein the cell prediction model is used for predicting cell positions and cell counts.

2. The cell counting method according to claim 1, wherein the performing one-to-one matching on the reference points corresponding to the cell image with the predicted cell points based on the cell labeling information and the predicted cell point information comprises:
calculating offsets between the predicted cell points and the reference points based on the predicted cell point position information, the reference point position information, and the confidence values in one-to-one correspondence with the predicted cell points, to obtain an offset matrix; and
minimizing the offsets between the reference points and the predicted cell points using a Hungarian algorithm based on the offset matrix, and determining the reference points in one-to-one correspondence with the predicted cell points.

3. The cell counting method according to claim 2, wherein the offsets between the predicted cell points and the reference points are calculated using the following formula:
$$D ( P , \hat{P} ) = {( τ {‖ {p}_{i} - ‖}_{2} - )}_{i ∈ N , j ∈ M}$$
wherein P represents the reference points, \hat{P} represents the predicted cell points, pᵢ represents the reference point position information of an i-th reference point, [symbol] represents the predicted cell point position information of a j-th predicted cell point, [symbol] represents the confidence value of the j-th predicted cell point, ∥·∥₂ represents a distance between the i-th reference point and the j-th predicted cell point, τ represents a weight, N represents a total number of the reference points, and M represents a total number of the predicted cell points.

4. The cell counting method according to claim 1, wherein the cell prediction model comprises:
a pre-trained convolutional neural network, a feature pyramid network, and a decoding network connected sequentially;
wherein an output end of the decoding network is further connected to a regression branch network and a classification branch network.

5. The cell counting method according to claim 4, wherein the inputting the cell image into the pre-constructed cell prediction model to obtain the predicted cell point information corresponding to the cell image comprises:
extracting first image features from the cell image using the pre-trained convolutional neural network;
performing feature upsampling and feature fusion on the first image features using the feature pyramid network to obtain second image features;
performing feature decoding on the second image features using the decoding network to generate a feature map; and
inputting the feature map into the regression branch network and the classification branch network, wherein the regression branch network is used for outputting the predicted cell point position information, and the classification branch network is used for outputting the confidence values in one-to-one correspondence with each predicted cell point.

6. A cell counting method, comprising:
acquiring an image of cells to be counted; and
inputting the image of cells to be counted into the cell prediction model of the cell counting method according to claim 1 to output a cell count in the image of cells to be counted and cell positions in one-to-one correspondence with cells.

7. A cell counting apparatus, comprising:
an acquisition module configured to acquire a cell image and cell labeling information in one-to-one correspondence with the cell image, wherein the cell labeling information comprises pre-labeled reference point position information and a cell count;
a prediction module configured to input the cell image into a pre-constructed cell prediction model to obtain predicted cell point information corresponding to the cell image, wherein the predicted cell point information comprises predicted cell point position information and confidence values in one-to-one correspondence with predicted cell points;
a matching module configured to perform one-to-one matching on reference points corresponding to the cell image with the predicted cell points based on the cell labeling information and the predicted cell point information to obtain a matching result; and
an optimization module configured to adjust the cell prediction model based on the matching result, wherein the cell prediction model is used for predicting cell positions and cell counts.

8. A computer device, comprising:
a memory and a processor, wherein the memory and the processor are communicatively connected to each other; the memory stores computer instructions, and the processor executes the computer instructions to perform the cell counting method according to claim 1.

9. The cell counting method according to claim 6, wherein the performing one-to-one matching on the reference points corresponding to the cell image with the predicted cell points based on the cell labeling information and the predicted cell point information comprises:
calculating offsets between the predicted cell points and the reference points based on the predicted cell point position information, the reference point position information, and the confidence values in one-to-one correspondence with the predicted cell points, to obtain an offset matrix; and
minimizing the offsets between the reference points and the predicted cell points using a Hungarian algorithm based on the offset matrix, and determining the reference points in one-to-one correspondence with the predicted cell points.

10. The cell counting method according to claim 9, wherein the offsets between the predicted cell points and the reference points are calculated using the following formula:
$$D ( P , \hat{P} ) = {( τ {‖ {p}_{i} - ‖}_{2} - )}_{i ∈ N , j ∈ M}$$
wherein P represents the reference points, \hat{P} represents the predicted cell points, pᵢ represents the reference point position information of an i-th reference point, [symbol] represents the predicted cell point position information of a j-th predicted cell point, [symbol] represents the confidence value of the j-th predicted cell point, ∥·∥₂ represents a distance between the i-th reference point and the j-th predicted cell point, τ represents a weight, N represents a total number of the reference points, and M represents a total number of the predicted cell points.

11. The cell counting method according to claim 6, wherein the cell prediction model comprises:
a pre-trained convolutional neural network, a feature pyramid network, and a decoding network connected sequentially;
wherein an output end of the decoding network is further connected to a regression branch network and a classification branch network.

12. The cell counting method according to claim 11, wherein the inputting the cell image into the pre-constructed cell prediction model to obtain the predicted cell point information corresponding to the cell image comprises:
extracting first image features from the cell image using the pre-trained convolutional neural network;
performing feature upsampling and feature fusion on the first image features using the feature pyramid network to obtain second image features;
performing feature decoding on the second image features using the decoding network to generate a feature map; and
inputting the feature map into the regression branch network and the classification branch network, wherein the regression branch network is used for outputting the predicted cell point position information, and the classification branch network is used for outputting the confidence values in one-to-one correspondence with each predicted cell point.

13. The computer device according to claim 8, wherein the performing one-to-one matching on the reference points corresponding to the cell image with the predicted cell points based on the cell labeling information and the predicted cell point information comprises:
calculating offsets between the predicted cell points and the reference points based on the predicted cell point position information, the reference point position information, and the confidence values in one-to-one correspondence with the predicted cell points, to obtain an offset matrix; and
minimizing the offsets between the reference points and the predicted cell points using a Hungarian algorithm based on the offset matrix, and determining the reference points in one-to-one correspondence with the predicted cell points.

14. The computer device according to claim 13, wherein the offsets between the predicted cell points and the reference points are calculated using the following formula:
$$D ( P , \hat{P} ) = {( τ {‖ {p}_{i} - ‖}_{2} - )}_{i ∈ N , j ∈ M}$$
wherein P represents the reference points, P represents the predicted cell points, pᵢ represents the reference point position information of an i-th reference point, [symbol] represents the predicted cell point position information of a j-th predicted cell point, [symbol] represents the confidence value of the j-th predicted cell point, ∥·∥₂ represents a distance between the i-th reference point and the j-th predicted cell point, τ represents a weight, N represents a total number of the reference points, and M represents a total number of the predicted cell points.

15. The computer device according to claim 8, wherein the cell prediction model comprises:
a pre-trained convolutional neural network, a feature pyramid network, and a decoding network connected sequentially;
wherein an output end of the decoding network is further connected to a regression branch network and a classification branch network.

16. The computer device according to claim 15, wherein the inputting the cell image into the pre-constructed cell prediction model to obtain the predicted cell point information corresponding to the cell image comprises:
extracting first image features from the cell image using the pre-trained convolutional neural network;
performing feature upsampling and feature fusion on the first image features using the feature pyramid network to obtain second image features;
performing feature decoding on the second image features using the decoding network to generate a feature map; and
inputting the feature map into the regression branch network and the classification branch network, wherein the regression branch network is used for outputting the predicted cell point position information, and the classification branch network is used for outputting the confidence values in one-to-one correspondence with each predicted cell point.
